(19) United States
(12) Patent Application Publication
(10) Pub. No.: US 2024/0247316 A1
(43) Pub. Date: Jul. 25, 2024

(54) METHOD FOR EARLY DETECTION, PREDICTION OF TREATMENT RESPONSE AND PROGNOSIS OF COLORECTAL CANCER

(71) Applicant: EG BIOMED CO., LTD., Taipei City (TW)

(72) Inventors: Ruo-Kai LIN, New Taipei City (TW); Chin-Sheng HUNG, Taipei City (TW); Sheng-Chao WANG, New Taipei City (TW); Shih-Yun LIN, New Taipei City (TW)

(21) Appl. No.: 17/997,490

(22) PCT Filed: Apr. 29, 2021
(86) PCT No.: PCT/US21/29965

§ 371 Date: Oct. 28, 2022

Related U.S. Application Data
(60) Provisional application No. 63/017,309, filed on Apr. 29, 2020.

(51) Int. Cl. C12Q 1/6886

(52) U.S. Cl. CPC C12Q 1/6886; C12Q 2600/154

(57) ABSTRACT

The present disclosure discloses a set of novel epigenetic biomarkers for early detection, prediction of treatment response and prognosis of colorectal cancer. Aberrant methylation of the epigenetic biomarkers can be detected in tumor tissues and plasma samples from colorectal cancer patients but not in normal individuals. The present disclosure also discloses primers and probes used herein.

This application is a 371 National Phase of International Patent Application No. PCT/US21/29965, filed on Apr. 29, 2021, which claims priority to and benefit of U.S. Provisional Patent Application No. 63/017,309, filed Apr. 29, 2020. The entirety of the aforementioned application is incorporated herein by reference.

The subject application contains a Sequence Listing which has been filed electronically in ASCII format and is hereby incorporated by reference in its entirety. Said ASCII copy, created on Apr. 28, 2022, is named G4590-08300NP_SeqListing_20221028.txt and is 4 kilobytes in size.

### FIELD OF THE INVENTION

The disclosure relates to epigenetic biomarkers for prediction of risk or susceptibility of colorectal cancer. Particularly, the disclosure provides a method for early detection, prediction of treatment response and prognosis of colorectal cancer based on methylation statuses of gene biomarkers.

### BACKGROUND OF THE INVENTION

Cancer is a group of diseases involving abnormal cell growth with the potential to invade or spread to other parts of the body and is a leading cause of deaths worldwide.

Methylated DNA has been studied as a potential class of biomarkers in the tissues of most tumor types. In many instances, DNA methyltransferases add a methyl group to DNA at cytosine-phosphate-guanine (CpG) island sites as an epigenetic control of gene expression.

US 20210003575 relates to the use of BMW Rep-protein as a biomarker for colon cancer. US 20200291479 provides methods for assessing the likelihood of effectiveness of chemotherapy such as oxaliplatin treatment in colorectal cancer patients as well as their survival prospect by determining the level of miR-133a in the cancer tissue. US 20200377959 discloses a method of detecting (e.g., screening for) colorectal cancer, the method comprising: determining a methylation status for each of the following, in deoxyribonucleic acid (DNA) of a human subject: (a) a methylation locus within gene ZNF132; (b) a first methylation locus within gene ADAMTS2; and (c) a second methylation locus within gene ADAMTS2; and diagnosing colorectal cancer in the human subject based on said determined methylation statuses.

However, the current techniques in detection of colorectal cancer are not satisfactory.

### SUMMARY OF THE INVENTION

The present disclosure discloses one or more novel epigenetic biomarkers for early detection, prediction of treatment response and prognosis of colorectal cancer. Aberrant methylation of the epigenetic biomarkers is detected in tumor tissues and plasma samples from cancer patients but not in normal individuals. The present disclosure also discloses primers and 5 probes used herein.

In one embodiment, the present disclosure provides a method for detecting a methylation status in a subject who is in a need of detecting a predisposition to colorectal cancer or predicting likelihood, treatment response, prognosis or recurrence of the colorectal cancer, comprising (a) providing a biological sample from the subject, and (b) determining a methylation status of a target DNA sequence comprising TMEM240 or a fragment thereof or MROH6 or a fragment thereof in the biological sample; wherein the presence of hypermethylation or hypomethylation in the target DNA sequence of the subject is indicative of colorectal cancer and/or indicative of the predisposition to, likelihood, poor treatment response, poor prognosis or recurrence of, colorectal cancer.

In one embodiment, the present disclosure provides a method for detecting a predisposition to colorectal cancer or predicting likelihood, treatment response, prognosis or recurrence of colorectal cancer in a subject, comprising (a) providing a biological sample from the subject, and (b) determining the methylation status of a target DNA sequence comprising TMEM240 or a fragment thereof or MROH6 or a fragment thereof in the biological sample, wherein the presence of hypermethylation or hypomethylation in the target DNA sequence of the subject is indicative of the predisposition to, likelihood, poor treatment response, poor prognosis or recurrence of, colorectal cancer.

In some embodiments, a target DNA sequence methylation specific probe or a target DNA sequence methylation specific primer is used to assay the methylation status of the target DNA sequence and the control DNA sequence in the biological sample.

In one embodiment, the presence of hypermethylation or hypomethylation in the target DNA sequence of the subject is determined by comparing the methylation status of the target DNA sequence to a methylation status of a control DNA sequence. In some embodiments, the present disclosure provides a method for detecting a predisposition to colorectal cancer or predicting likelihood, treatment response, prognosis or recurrence of colorectal cancer in a subject, comprising (a) providing a biological sample from a subject comprising a target DNA sequence comprising TMEM240 or a fragment thereof or MROH6 or a fragment thereof, and (b) determining methylation statuses of the target DNA sequence and of a control DNA sequence in the biological sample using a target DNA sequence methylation specific probe or a target DNA sequence methylation specific primer, (c) measuring a relative methylation status of the target DNA sequence compared to a control DNA sequence, (d) identifying the subject as having the predisposition to, likelihood, poor treatment response, poor prognosis or recurrence of, colorectal cancer when hypermethylation or hypomethylation is present in the relative methylation status. In some embodiments, the hypermethylation described herein is indicated when the methylation status in TMEM240 or a fragment thereof is about 30-fold, about 32-fold, about 34-fold, about 35-fold, about 36-fold, about 37-fold, about 38-fold, about 39-fold or about 40-fold higher than that of the control DNA sequence. In a further embodiment, the methylation status in the TMEM240 or a fragment thereof that is about 37.5-fold higher or lower than that of the control DNA sequence indicates colorectal cancer. In some embodiments, the hypermethylation described herein is indicated when the methylation status in MROH6 or a fragment thereof is about 35-fold, about 37-15 fold, about 39-fold, about 40-fold, about 41-fold, about 42-fold, about 43-fold, about 44-fold, about 45-fold, about 46-fold, about 47-fold, or about 48-fold higher than that of the control DNA sequence. In a further embodiment, the methylation status in the MROH6 or a fragment thereof that is about 44-fold higher or lower than that of the control DNA sequence indicates colorectal cancer. In a further embodiment, the control DNA sequence is in a normal tissue.

In some embodiments, the biological sample described herein is a tissue, cell, blood, urine, serum, plasma, stool, ascites, sputum, saliva, gastric juice, bile, or oral mucosa.

In some embodiments, the methylation status is detected by a polymerase chain reaction, nucleic acid sequencing (such as bisulfite sequencing or pyrosequencing), bisulfite conversion, mass spectrometry, methylation specific nuclease, mass-based separation, target capture or microarray. In a particular embodiment, the methylation status is detected by a polymerase chain reaction.

In some embodiments, the hypermethylation is indicated when a Cₜ value of the polymerase chain reaction for determining the methylation status of TMEM240 or a fragment thereof in the human subject is less than 40, 41, 42, 43, 44, 45, 46, 47, 48, 49, or 50. In some embodiments, the hypermethylation is indicated when a Cₜ value of the polymerase chain reaction for determining the methylation status of MROH6 or a fragment thereof in the human subject is less than 35, 36, 37, 38, 39, 40, 41, 42, 43, 44, or 45. In a preferred embodiment of the disclosure, the hypermethylation is indicated when a Cₜ value of the polymerase chain reaction for determining the methylation status of TMEM240 or a fragment thereof in the human subject is less than 45; or the hypermethylation is indicated when a Cₜ value of the polymerase chain reaction for determining the methylation status of MROH6 or a fragment thereof in the human subject is less than 40.

In some embodiments, the method described herein is for detecting a methylation status in a human subject who has a need of detection of colorectal cancer.

Certain embodiments of the target DNA sequence methylation specific primer used in the methylation determination for TMEM240 or a fragment thereof has an identity of about 85%, about 86%, about 87%, about 88%, about 89%, about 90%, about 91%, about 92%, about 93%, about 94%, about 95%, about 96%, about 97%, about 98%, about 99%, or higher percent to a sequence(s) selected from the group consisting of SEQ ID NOs: 1, 2, or 3. Certain embodiments of the target DNA sequence methylation specific probe used in the methylation determination for TMEM240 or a fragment thereof has an identity of about 85%, about 86%, about 87%, about 88%, about 89%, about 90%, about 91%, about 92%, about 93%, about 94%, about 95%, about 96%, about 97%, about 98%, about 99%, or higher percent to a sequence(s) selected from the group consisting of SEQ ID NO: 4. In some embodiments, the target DNA sequence methylation specific primer for used in the methylation determination for MROH6 or a fragment thereof has a sequence with identity of at least 85%, about 86%, about 87%, about 88%, about 89%, about 90%, about 91%, about 92%, about 93%, about 94%, about 95%, about 96%, about 97%, about 98%, about 99% to SEQ ID NOs: 5, or 6. In some embodiments, the target DNA sequence methylation specific probe for used in the methylation determination for MROH6 or a fragment thereof has a sequence with identity of at least 85%, about 86%, about 87%, about 88%, about 89%, about 90%, about 91%, about 92%, about 93%, about 94%, about 95%, about 96%, about 97%, about 98%, about 99% to SEQ ID NO: 7. In some embodiments, the target DNA sequence methylation specific probe for used in the methylation determination for TMEM240 or a fragment thereof has a sequence with identity of at least 85% to a sequence selected from the group consisting of SEQ ID NOs: 1, 2, and 3. In some embodiments, the target DNA sequence methylation specific probe for TMEM240 or a fragment thereof has an identity of about 85% to a sequence of SEQ ID NO: 4. In some embodiments, the target DNA sequence methylation specific primer for MROH6 or a fragment thereof has an identity of about 85% to a sequence selected from the group consisting of SEQ ID NOs: 5, and 6. In some embodiments, the target DNA sequence methylation specific probe for MROH6 or a fragment thereof has an identity of about 85% to a sequence of SEQ ID NO: 7.

In a further embodiment, the target DNA sequence further comprises one or more DNA sequences selected from the group consisting of BEND5 or a fragment thereof and SMAD3 or a fragment thereof, or any of combinations thereof.

Certain embodiments of the target DNA sequence described herein includes any of the following combinations of the DNA sequences: TMEM240 or a fragment thereof and MROH6 or a fragment thereof; TMEM240 or a fragment thereof, MROH6 or a fragment thereof and BEND5 or a fragment thereof; TMEM240 or a fragment thereof, MROH6 or a fragment thereof, BEND5 or a fragment thereof and SMAD3 or a fragment thereof; TMEM240 or a fragment thereof, BEND5 or a fragment thereof and SMAD3 or a fragment thereof; TMEM240 or a fragment thereof, MROH6 or a fragment thereof, and SMAD3 or a fragment thereof; MROH6 or a fragment thereof and BEND5 or a fragment thereof; and MROH6 or a fragment thereof, BEND5 or a fragment thereof and SMAD3 or a fragment thereof. In a further embodiment, the target DNA sequences comprise TMEM240 or a fragment thereof and MROH6 or a fragment thereof.

In some embodiments, the methylation status is determined by a polymerase chain reaction, and the hypermethylation is indicated when a Cₜ value of the polymerase chain reaction for determining the methylation status of BEND5 or a fragment thereof in the human subject is less than 41, 42, 43, 44, 45, 46, 47, 48, 49, or 50. In some embodiments, the methylation status is determined by a polymerase chain reaction, and the hypomethylation is indicated when a Cₜ value of the polymerase chain reaction for determining the methylation status of SMAD3 or a fragment thereof in the human subject is higher than 30, 31, 32, 33, 34, 35, 36, 37, 38, 39, or 40. In some embodiments of the disclosure, the hypermethylation is indicated when a Cₜ value of the polymerase chain reaction for determining the methylation status of BEND5 or a fragment thereof in the human subject is less than 45. In some embodiments, the hypomethylation is indicated when a Cₜ value of the polymerase chain reaction for determining the methylation status of SMAD3 or a fragment thereof in the human subject is higher than 45.

In some embodiments of the disclosure, the method further comprising a step of defining a score as 1 if the presence of hypermethylation or hypomethylation of each gene or a fragment thereof and, defining a score as 0 if the absence of hypermethylation or hypomethylation of each gene or a fragment thereof, and summing the score.

In some embodiments of the disclosure, the method further comprises:
defining a score for TMEM240 as 1, if a Cₜ value of TMEM240 or a fragment thereof is less than 45; defining a score for TMEM240 as 0, if a Cₜ value of TMEM240 or a fragment thereof is higher than or equal to 45;
defining a score for MROH6 as 1, if a Cₜ value of MROH6 or a fragment thereof is less than 40;
defining a score for MROH6 as 0, if a Cₜ value of MROH6 or a fragment thereof is higher than or equal to 45;
defining a score for BEND5 as 1, if a Cₜ value of BEND5 or a fragment thereof is less than 45, and summing the score; defining a score for BEND5 as 0, if a Cₜ value of BEND5 or a fragment thereof is higher than or equal to 45; or
defining a score for SMAD3 as 1, if a Cₜ value of SMAD3 or a fragment thereof is higher than 45;
defining a score for SMAD3 as 0, if a Cₜ value of SMAD3 or a fragment thereof is less than or equal to 45; and
summing the scores for TMEM240, MROH6, BEND5 and SMAD3.

In some embodiments of the disclosure, if the sum of the scores for TMEM240, MROH6, and BEND5 is higher than 0.15, 0.16, 0.17, 0.18, 0.19, 0.20, 0.21, 0.22, 0.23, 0.24, or 0.25, the subject is indicative of the predisposition to, likelihood, poor treatment response, poor prognosis or recurrence of, colorectal cancer. In some embodiments of the disclosure, if the sum of the scores for TMEM240, MROH6, and BEND5 is higher than 0.20, the subject is indicative of the predisposition to, likelihood, poor treatment response, poor prognosis or recurrence of, colorectal cancer.

In some embodiments, a target DNA sequence methylation specific probe or a target DNA sequence methylation specific primer or any of combinations thereof, as described herein, are further used for determining a methylation status of the following one or more DNA sequences or any of combinations thereof: BEND5 or a fragment thereof and SMAD3 or a fragment thereof.

In some embodiments, the target DNA sequence methylation specific primer for BEND5 or a fragment thereof described herein has a sequence with identity of at least 85%, 88%, 90%, 91%, 92%, 93%, 94%, 95%, 96%, 97%, 98% or 99% to SEQ ID NO: 8, 9, 10 or 11. In some further embodiments, the target DNA sequence methylation specific primer for BEND5 or a fragment thereof has a sequence of SEQ ID NO: 8, 9, 10 or 11. The target DNA sequence methylation specific probe for BEND5 or a fragment thereof has a sequence with identity of at least 85%, 88%, 90%, 91%, 92%, 93%, 94%, 95%, 96%, 97%, 98% or 99% to SEQ ID NO: 12 or 13. In a further embodiment, the target DNA sequence methylation specific probe for BEND5 or a fragment thereof has a sequence of SEQ ID NO: 12 or 13.

In some embodiments, the target DNA sequence methylation specific primer for SMAD3 or a fragment thereof described herein has a sequence with identity of at least 85%, 88%, 90%, 91%, 92%, 93%, 94%, 95%, 96%, 97%, 98% or 99% to SEQ ID NO: 14 or 15. In some further embodiments, the target DNA sequence methylation specific primer for SMAD3 or a fragment thereof has a sequence of SEQ ID NO: 14 or 15. The target DNA sequence methylation specific probe for SMAD3 or a fragment thereof has a sequence with identity of at least 85%, 88%, 90%, 91%, 92%, 93%, 94%, 95%, 96%, 97%, 98% or 99% to SEQ ID NO: 16. In a further embodiment, the target DNA sequence methylation specific probe for SMAD6 or a fragment thereof has a sequence of SEQ ID NO: 16.

In a further embodiment, determining the methylation status further comprises a step of measuring the specificity and sensitivity by a weighted sum score analysis. In some further embodiments, determining the methylation status of the combination of target TMEM240, MROH6, BEND5 and SMAD3 or a fragment thereof or target TMEM240, MROH6, BEND5 and SMAD3 or a fragment thereof has about 100% of sensitivity and about 100% specificity and about 100% of accuracy.

In a further embodiment, the method described herein further comprises a step of administering an anti-colorectal cancer agent to the subject.

The present disclosure provides a method for detecting a predisposition to colorectal cancer or predicting likelihood, treatment response, prognosis or recurrence of colorectal cancer in a subject, comprising (a) providing a biological sample from the subject, and (b) determining a methylation status of a target DNA sequence comprising TMEM240 or a fragment thereof and MROH6 or a fragment thereof in the biological sample; wherein the presence of hypermethylation or hypomethylation in the target DNA sequence of the subject is indicative of the predisposition to, likelihood, poor treatment response, poor prognosis or recurrence of, colorectal cancer.

In some embodiments of the disclosure, the method comprises determining the methylation status of TMEM240 or a fragment thereof using a target DNA sequence methylation specific primer having a sequence with identity of at least 85% to SEQ ID NO: 1, 2, or 3 or a target DNA sequence methylation specific probe having a sequence with identity of at least 85% to SEQ ID NO: 4, and determining a methylation status of MROH6 or a fragment thereof using a MROH6 methylation specific primer having a sequence with homology of at least 85% to SEQ ID NO: 5 or 6 or a MROH6 sequence methylation specific probe having a sequence with homology of at least 85% to SEQ ID NO: 7.

In some embodiments of the disclosure, the target DNA sequence further comprises one or more DNA sequences selected from the group consisting of BEND5 or a fragment thereof and SMAD3 or a fragment thereof, or any of combinations thereof.

The present disclosure provides an isolated nucleic acid molecule having a sequence selected from the group consisting of: SEQ ID Nos: 1 to 16.

The present disclosure also provides a kit for detecting a predisposition to colorectal cancer or predicting likelihood, treatment response, prognosis or recurrence of colorectal cancer in a subject, which comprises an isolated nucleic acid molecule for assaying a methylation status of the target DNA sequence as described herein. The kit may further comprise sodium bisulfite and adapters for whole target genes amplification, and polynucleotides (e.g., detectably-labeled polynucleotides) to quantify the presence of a methylated and/or an unmethylated cytosine residue in the target DNA sequence in target DNA sequence as described herein. Furthermore, the kit may further comprise a methylation sensing restriction enzyme for whole target sequence or genes amplification.

The present disclosure provides a target DNA sequence methylation specific primer pair for detection of a methylation status of TMEM240 or a fragment thereof, comprising SEQ ID NOs: 1 and 2 or a sequence having at least 85% identity thereof; or SEQ ID NOs: 1 and 3 or a sequence having at least 85% identity thereof. The present disclosure provides a target DNA sequence methylation specific probe for detection of a methylation status of TMEM240 or a fragment thereof, comprising SEQ ID NO: 4 or a sequence having at least 85% identity thereof.

The present disclosure provides a target DNA sequence methylation specific primer pair for detection of a methylation status of MROH6 or a fragment thereof, comprising SEQ ID NOs: 5 and 6 or a sequence having at least 85% identity thereof. The present disclosure provides a target DNA sequence methylation specific probe for detection of a methylation status of MROH6 or a fragment thereof, comprising SEQ ID NO: 7 or a sequence having at least 85% identity thereof.

The present disclosure provides a target DNA sequence methylation specific primer pair for detection of a methylation status of BEND5 or a fragment thereof, comprising SEQ ID NOs: 8 and 9 or a sequence having at least 85% identity thereof; or SEQ ID NOs: 10 and 11 or a sequence having at least 85% identity thereof. The present disclosure provides a target DNA sequence methylation specific probe for detection of a methylation status of BEND5 or a fragment thereof, comprising SEQ ID NO: 12 or 13 or a sequence having at least 85% identity thereof.

The present disclosure provides a target DNA sequence methylation specific primer pair for detection of a methylation status of SMAD3 or a fragment thereof, comprising SEQ ID NOs: 14 and 15 or a sequence having at least 85% identity thereof. The present disclosure provides a target DNA sequence methylation specific probe for detection of a methylation status of SMAD3 or a fragment thereof, comprising SEQ ID NO: 16 or a sequence having at least 85% identity thereof.

The present disclosure also discloses a kit for detecting a predisposition to colorectal cancer or predicting likelihood, treatment response, prognosis or recurrence of colorectal cancer in a subject, which comprises a target DNA sequence methylation specific primer pair for detection of a methylation status of a target DNA sequence comprising TMEM240 or a fragment thereof and MROH6 or fragment thereof. In one embodiments of the disclosure, the kit further comprises a target DNA sequence methylation specific probe for detection of a methylation status of a target DNA sequence comprising TMEM240 or a fragment thereof and MROH6 or fragment thereof.

In some embodiments, the kit further comprises one or more target DNA sequence methylation specific primer pairs for detection of a methylation status of BEND5 or a fragment thereof or SMAD3 or a fragment thereof, or any combination thereof. In some embodiments, the kit further comprises one or more target DNA sequence methylation specific probe for detection of a methylation status of BEND5 or a fragment thereof or SMAD3 or a fragment thereof, or any combination thereof.

### BRIEF DESCRIPTION OF THE DRAWINGS

FIGS. 1A to 1H show the heatmap for difference of methylation statuses of target nucleic acid and promoter, exon and gene body region of genes between tumor tissues and adjacent normal tissues, respectively (FIG. 1A: TMEM240 of Taiwan samples; FIG. 1B: MROH6 of Taiwan samples; FI. 1C: BEND5 of Taiwan samples; FIG. 1D: SMAD3 of Taiwan samples; FIG. 1E: TMEM240 of TCGA samples; FIG. 1F: MRTH6 of TCGA samples; FIG. 1G: BEND5 of TCGA samples; FIG. 1H: SMAD3 of TCGA samples).

FIGS. 2A to 2E shows the difference in early detection of the methylation status of epigenetic biomarkers of target genes in plasma samples of healthy subjects and colorectal cancer patients (FIG. 2A: TMEM240; FIG. 2B: MROH6; FIG. 2C: BEND5; FIG. 2D: SMAD3; FIG. 2E: the DNA methylation levels forTMEM240; MROH6 and BEND5).

FIG. 3 shows the sum of DNA methylation levels (scores) of epigenetic biomarkers of target genes in plasma samples of healthy subjects and colorectal cancer patients.

FIG. 4 shows Receiver Operating Characteristic (ROC) Curve Analysis exhibiting the early detection of the methylation status of epigenetic biomarkers of target genes in colorectal cancer patients and healthy subjects.

### DETAILED DESCRIPTION OF THE INVENTION

It is understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise. Thus, for example, reference to “a biomarker” includes a mixture of two or more biomarkers, and the like.

The term “AUC” as used herein is an abbreviation for the area under a curve. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better, optimum is 1, a random test would have a ROC curve lying on the diagonal with an area of 0.5; for reference: J. P. Egan. Signal Detection Theory and ROC Analysis, Academic Press, New York, 1975).

The term “a biological sample” refers to a sample of tissue, cells, or fluid isolated from a subject, including but not limited to, for example, blood, buffy coat, plasma, serum, blood cells (e.g., peripheral blood mononucleated cells (PBMCS), band cells, neutrophils, metamyelocytes, monocytes, or T cells), fecal matter, urine, bone marrow, bile, stool, ascites, sputum, spinal fluid, lymph fluid, samples of the skin, external secretions of the skin, respiratory, intestinal, and genitourinary tracts, tears, saliva, milk, organs, biopsies and also samples of in vitro cell culture constituents, including, but not limited to, conditioned media resulting from the growth of cells and tissues in culture medium, e.g., recombinant cells, and cell components.

The term “a biomarker” refers to a nucleic acid molecule which is present in a sample taken from patients having human cancer as compared to a comparable sample taken from control subjects (e.g., a person with a negative diagnosis or undetectable cancer, normal or healthy subject). The biomarker can be a nucleic acid, a fragment of a nucleic acid, a polynucleotide, or an oligonucleotide that can be detected and/or quantified. Biomarkers include polynucleotides comprising nucleotide sequences from genes.

The term “a CpG island” as used herein refers to stretches of DNA in a genome that are rich in GC relative to the rest of the genome. Typically, the GC content is 50% or greater in these regions, which extend over hundreds of base pairs and sometimes thousands. Often these regions mark the 5′ ends of genes.

As used herein, the term “early detection” of cancer refers to discovering the likelihood of cancer before metastasis. Preferably, it refers to discovering the likelihood of cancer before a morphological change in a sample tissue or cell is observed.

As used herein, the terms “detect”, “detecting” or “detection” may describe either the general act of discovering or discerning or the specific observation of a detectably labeled composition.

The term “gene” refers to a nucleic acid (e.g., DNA) sequence that comprises coding sequences necessary for the production of a polypeptide, precursor, or RNA (e.g., non-coding RNAs such as ribosomal RNA, transfer RNA, splicosomal RNA, microRNA). A polypeptide or non-coding RNA can be encoded by a full-length coding sequence or by any portion of the coding sequence so long as the desired activity or functional properties (e.g., enzymatic activity, ligand binding, signal transduction, immunogenicity, etc.) of the full-length or fragment polypeptide are retained. Accordingly, a gene can include or exclude promoter sequences, terminators, translational regulatory sequences such as ribosome binding sites and internal ribosome entry sites, enhancers, silencers, insulators, boundary elements, replication origins, matrix attachment sites and locus control regions. The term also encompasses the coding region of a structural gene and the sequences located adjacent to the coding region on both the 5′ and 3′ ends for a distance of about 1 kb or more on either end such that the gene corresponds in length to the full-length mRNA. The term “gene” further includes both cDNA and genomic forms of a gene.

As used herein, the term “promoter” refers to a region of DNA that generally is located upstream (towards the 5′ region of a gene) of a gene and is needed to initiate and drive transcription of the gene. A promoter may permit proper activation or repression of a gene that it controls. A promoter may contain specific sequences that are recognized by transcription factors. These factors may bind to a promoter DNA sequence, which results in the recruitment of RNA polymerase, an enzyme that synthesizes RNA from the coding region of the gene. The promoter generally refers to all gene regulatory elements located upstream of the gene, including, upstream promoters, 5′ UTR, introns, and leader sequences.

The term “exon” refers to any segment of an interrupted gene that is represented in a mature RNA product. The term “intron” refers to any segment of DNA that is transcribed but removed from within the transcript by splicing together the exons on either side of it. Operationally, exon sequences occur in the mRNA sequence of a gene. Operationally, intron sequences are the intervening sequences within the genomic DNA of a gene, bracketed by exon sequences and usually having GT and AG splice consensus sequences at their 5′ and 3′ boundaries.

As used herein, the term “homology” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In some embodiments, the degree of homology between the two sequences is sufficient to allow homologous recombination therebetween, under appropriate stringent conditions.

Techniques for determining nucleic acid and amino acid sequence identity include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100.

In some embodiments, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. See, e.g., Sambrook et al., supra; Nucleic Acid Hybridization: A Practical Approach, editors B. D. Hames and S. J. Higgins, (1985) Oxford; Washington, D.C.; IRL Press).

As used herein, the term “prediction” refers to the likelihood that a patient will respond either favorably or unfavorably to a drug or set of drugs, and also the extent of those responses. Thus, treatment predictive factors are variables related to the response of an individual patient to a specific treatment, independent of prognosis.

The term “methylation,” as used herein, refers to the presence of a methyl group added by the action of a DNA methyl transferase enzyme to a cytosine base or bases in a region of nucleic acid, e.g., genomic DNA.

The term “methylation status” of a nucleic acid molecule refers to the presence or absence of one or more methylated nucleotide bases in the nucleic acid molecule. For example, a nucleic acid molecule containing a methylated cytosine is considered methylated (i.e., the methylation state of the nucleic acid molecule is methylated). A nucleic acid molecule that does not contain any methylated nucleotides is considered unmethylated.

The term “hypermethylation” refers to the average methylation state corresponding to an increased presence of methylated nucleotide bases in the nucleic acid molecule at one or a plurality of CpG dinucleotides within a DNA sequence of a test DNA sample, relative to the amount of methylated nucleotide bases in the nucleic acid molecule found at corresponding CpG dinucleotides within a normal control DNA sample.

The term “hypomethylation” refers to the average methylation state corresponding to a decreased presence of methylated nucleotide bases in the nucleic acid molecule at one or a plurality of CpG dinucleotides within a DNA sequence of a test DNA sample, relative to the amount of methylated nucleotide bases in the nucleic acid molecule found at corresponding CpG dinucleotides within a normal control DNA sample.

The term “a Cₜ value” is the abbreviation for threshold cycle and is defined as the calculated cycle number at which the PCR product crosses a threshold of detection.

The term “subject” refers to humans.

The term “susceptibility” refers to a constitution or condition of the body which makes the tissues react in special ways to certain extrinsic stimuli and thus tends to make the individual more than usually susceptible to certain diseases.

The term “target site” or “target sequence” refers to a nucleic acid sequence that defines a portion of a nucleic acid to which a binding molecule will bind, provided sufficient conditions for binding exist.

The term “risk” refers to the estimated chance of getting a disease during a certain time period, such as within the next 10 years, or during the subject's lifetime.

The term “prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease.

The term “weight sum score” refers to every possible alternative being rated by a score including all objectives, individually weighted to stress the importance of different objectives.

As used herein, the term “nucleic acid molecule” (or “nucleic acid” or “polynucleotide”) may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide”. A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

Cancer is characterized by an abnormal growth of a cell caused by one or more mutations or modifications of a gene leading to dysregulated balance of cell proliferation and cell death. In many disease processes, such as cancer, gene promoter CpG islands acquire abnormal hypermethylation, which results in transcriptional silencing that can be inherited by daughter cells following cell division. DNA methylation causing silencing in cancer typically occurs at multiple CpG sites in the CpG islands that are present in the promoters of protein coding genes. Alterations of DNA methylation have been recognized as an important component of cancer development. DNA methylation profiling provides higher clinical sensitivity and dynamic range compared to other cancer detections. Accordingly, the present disclosure provides a method and kit for early prediction, treatment response and prognosis or recurrence monitoring of colorectal cancer.

In the present disclosure, the methylation status of target DNA sequences or a fragment thereof in a biological sample are measured to detect colorectal cancer or detect a predisposition to colorectal cancer or predict treatment response, prognosis or recurrence of colorectal cancer in a human subject. In further embodiments, the methylation status of a target DNA sequence comprising TMEM240 or a fragment thereof or MROH6 or a fragment thereof in a biological sample are measured to detect colorectal cancer or detect a predisposition to colorectal cancer or predict treatment response, prognosis or recurrence of colorectal cancer in a human subject. In a further embodiment, the methylation status of BEND5 or a fragment thereof or SMAD3 or a fragment thereof is further measured.

TMEM240 encodes a transmembrane domain-containing protein transmembrane protein 240, found in the brain and cerebellum. Mutations of TMEM240 were found to cause spinocerebellar ataxia 21 (SCA21) with mental retardation, severe cognitive impairment, and hypokinetic and hyperkinetic movement disorders. In one preferred embodiment, the target DNA sequence comprises the promoter and exon 1 regions of TMEM240.

MROH6 gene encodes maestro heat like repeat family member 6. Diseases associated with MROH6 include Non-Syndromic Intellectual Disability and Autosomal Recessive Non-Syndromic Intellectual Disability.

BEND5 gene encodes BEN Domain Containing 5, which acts as a transcriptional repressor. In one preferred embodiment, the target DNA sequence comprises the promoter and exon 1 regions of BEND5.

SMAD3 gene encodes SMAD Family Member 3, which is related to the transforming growth factor-β. In one preferred embodiment, the target DNA sequence comprises the promoter 5 regions of SMAD3.

In some embodiments, the methylation comprises a cytosine methylation site. In some instances, cytosine methylation comprises 5-methylcytosine (5-mCyt) and 5-hydroxymethylcytosine. In some cases, a cytosine methylation site occurs in a CpG dinucleotide motif. In other cases, a cytosine methylation site occurs in a CHG or CHH motif, in which is adenine, cytosine or thymine. In some instances, one or more CpG dinucleotide motif or CpG site forms a CpG island, a short DNA sequence rich in CpG dinucleotide. In some instances, CpG islands are typically, but not always, between about 0.2 to about 1 kb in length. In some instances, the methylation comprises CpG island methylation.

In some embodiments, the methylation status is analyzed by a methylation specific enzymatic digest; bisulfite sequencing; an analysis selected from promoter methylation, CpG island methylation, MSP, HeavyMethyl, MethyLight, and Ms-SNuPE; and other methods relying on a detection of amplified DNA. The term “MethyLight™” refers to a fluorescence-based real-time PCR technique. MethylLight is described by Eads et al., Cancer Res. 59:2302-2306, 1999, herein incorporated by reference.

The term “HeavyMethyl” assay, refers to an assay wherein methylation specific blocking probes (also referred to herein as blockers) covering CpG positions between, or covered by the amplification primers enable methylation-specific selective amplification of a nucleic acid sample.

The term “Ms-SNuPE” refers to Methylation-sensitive Single Nucleotide Primer Extension. MsSNuPE is described by Gonzalgo & Jones, Nucleic Acids Res. 25:2529-2531, 1997, herein incorporated by reference.

The term “MSP” refers to Methylation-specific PCR. MSP is described by Herman et al. Proc. Natl. Acad. Sci. USA 93:9821-9826, 1996, and by U.S. Pat. No. 5,786,146, each of which are herein incorporated by reference.

Bisulfite modification of DNA is a method to assess CpG methylation status. 5-methylcytosine is the most frequent covalent base modification in the DNA of eukaryotic cells. However, 5-methylcytosine positions cannot be identified directly by sequencing or hybridization methods, because 5-methylcytosine has the same base pairing behavior as cytosine. Moreover, the epigenetic information carried by 5-methylcytosine is completely lost during, e.g., PCR amplification. Bisulfite sequencing is a method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. However, 5-methylcytosine remains unmodified under the aforementioned conditions. Thus, the original DNA is converted in such a manner that methylcytosine, which originally could not be distinguished from cytosine by its hybridization behavior, can be detected as the only remaining cytosine using molecular biological techniques, for example, by amplification and hybridization, or by sequencing.

In one embodiment, the methylation status is detected by polymerase chain reaction, nucleic acid sequencing (such as bisulfite sequencing or pyrosequencing), bisulfite conversion, mass spectrometry, methylation specific nuclease, mass-based separation, target capture or microarray. In one embodiment, the methylation status is detected by using primers to amplify a methylated CpG of the target genes. In a further embodiment, the detection of methylation is conducted by PCR, methylation specific PCR (MSP), real-time methylation specific PCR, quantitative methylation-specific PCR (QMSP), PCR using a methylated DNA-specific binding protein or quantitative PCR.

In one embodiment of the present disclosure, a target DNA sequence methylation specific primer that could amplify a methylated CpG of the genes described herein might be used. The target DNA sequence methylation specific primer comprises at least one or more CpG dinucleotide in a region which hybridizes to the methylated CpG of the genes. Specifically, the target DNA sequence methylation specific primer for amplifying a methylated CpG of the genes comprise sequence having a homology of about 85%, about 86%, about 87%, about 88%, about 89%, about 90%, about 91%, about 92%, about 93%, about 94%, about 95%, about 96%, about 97%, about 98%, about 99%, or higher percent to sequence(s) selected from the group consisting of the following sequences as shown in Table 1.

In one embodiment of the present disclosure, a target DNA sequence methylation specific probe capable of hybridizing with a methylated CpG of the genes described herein might be used. The target DNA sequence methylation specific probe capable of hybridizing with a methylated CpG of the genes comprise at least one or more CpG dinucleotide in a region which hybridizes to the methylated CpG of the genes. Specifically, probe(s) might comprise sequence(s) having a homology of about 85%, about 86%, about 87%, about 88%, about 89%, about 90%, about 91%, about 92%, about 93%, about 94%, about 95%, about 96%, about 97%, about 98%, about 99%, or higher percent to sequence(s) selected from the group consisting of the following sequences as shown in Table 1.

TABLE 1

| SEQ ID NO. | Primer or Probe | Sequence |
|---|---|---|
| 1 | TMEM240-qMSP-F | TTTAGAATTATGAAGATTATGGTGTTC |
| 2 | TMEM240-qMSP-R | AAAACTCAACATCGAACCGA |
| 3 | TMEM240-qMSP-R2 | CGACCCCGCCCGATATCCATAA |
| 4 | TMEM240-qMSP- | TTTAGAATTATGAAGATTATGGTGTTC |
| | probe | |
| 5 | MROH6-qMSP-F2 | GGTGAGTTTTTGATTTGTAATTGTC |
| 6 | MROH6-qMSP-R | ATCTCGTACCGCTACTACTACGC |
| 7 | MROH6-qMSP-probe | GTCGGGGGTTGTTGATTTTAGTAGCGTT |
| 8 | BEND5-qMSP-F2 | GTTTGGGTTTTGGGGAGTC |
| 9 | BEND5-qMSP-R | GATCGAACAACTCAACCCG |
| 10 | BEND5-forward | GTT TTT GTG CGG TTT TTG GA |
| 11 | BEND5-reverse | AAC CGC GAA CGA AAA CTA AA |
| 12 | BEND5-qMSP-probe | CGAAAATAAAAATCCGACGA |
| 13 | BEND5-probe | TT GTT ACG CG TTG TTC GTG T |
| 14 | SMAD3-qMSP-F | GAATAAGGTCGTTAGTTATTATCGT |
| 15 | SMAD3-qMSP-R | AATCAAATCTACCCGAATCGAA |
| 16 | SMAD3-qMSP-probe | GAAAGAAAGAAAGAAAGTAAATTTTATTTTTA |
| | | AGCG |

In one embodiment, the detection of the methylation status of the target DNA sequence comprises the presence of hypermethylation in the target DNA sequence relative to a normal state of the target genes.

In some embodiments, the biological sample is a tissue, cell, blood, urine, serum, plasma, stool, ascites, sputum, saliva, gastric juice, bile, or oral mucosa from a human subject suspected of having colorectal cancer or a human subject to be detected.

As used herein, the term “who is in a need of detection of cancer” refers to an individual who has received an initial diagnosis (e.g., a CT scan showing a mass or increased biomarker level) but for whom the stage of cancer or presence or absence of methylated genes indicative of cancer is not known. The term further includes people who once had cancer (e.g., an individual in remission).

In some embodiments, a detection test to correctly predict status is measured as the sensitivity of the assay, the specificity of the assay or the area under a receiver operated characteristic (ROC) curve (AUC). The greater the area under the ROC curve, for example, the more accurate or powerful the predictive value of the test.

In one embodiment, a weighted sum score is measured to determine the methylation status in the nucleic acid sequence and genes as an indicator. The weighted sum model (WSM) is the best known and simplest multi-criteria decision analysis (MCDA)/multi-criteria decision making method for evaluating a number of alternatives in terms of a number of decision criteria. According to the present disclosure, the weighted sum score analysis shows the combination of TMEM240, MROH6, BEND5 and SMAD3 shows a sensitivity of about 100% and a specificity of about 96% than the control.

In some embodiments, one or more of the biomarkers disclosed herein show a statistical difference in different samples of at least p<0.05. Detection tests that use these biomarkers may show an AUC of at least 0.9.

In some embodiments, the hypermethylation status of the epigenetic biomarkers in DNA sequences described herein correlates with a “poor” prognosis or the likelihood that a subject will likely respond unfavorably to a drug or set of drugs, leading to a progression of a cancer and/or to refractory of one or more therapeutic agents. In some instances, a “poor” prognosis refers to the likelihood that a subject will not respond to a drug or set of drugs, leading to a progression of a cancer. In some instances, a “poor” prognosis refers to the survival of a subject of from less than 5 years to less than 1 month. In some instances, a “poor” prognosis refers to the survival of a subject in which the survival of the subject upon treatment is from less than 5 years to less than 1 month. In some instances, a “poor” prognosis further refers to the likelihood that a subject will develop a refractory cancer toward one or more drugs.

In some embodiments, the present disclosure provides an isolated nucleic acid molecule having a sequence selected from the group consisting of: SEQ ID Nos: 1 to 16.

In some embodiments, the present disclosure provides a kit for detecting a methylation status in a human subject who is in a need of detecting a predisposition to colorectal cancer or predicting likelihood, treatment response, prognosis or recurrence of colorectal cancer in a human subject, which comprises an isolated nucleic acid molecule having a sequence selected from the group consisting of: SEQ ID Nos: 1 to 4 or 5 to 7 for assaying a methylation status of a target DNA sequence comprising TMEM240 or a fragment thereof or MROH6 or a fragment thereof.

In some preferred embodiments of the disclosure, the target DNA sequence further comprises one or more DNA sequences selected from the group consisting of BEND5 or a fragment thereof and SMAD3 or a fragment thereof, or any of combinations thereof, and the kit further comprises an isolated nucleic acid molecule having a sequence selected from the group consisting of: SEQ ID Nos: 8 to 16 for assaying a methylation status of the target DNA sequence.

In some instances, the kit comprises a plurality of the target DNA sequence methylation specific primers or target DNA sequence methylation specific probes to detect or measure the methylation status/levels of one or more target DNA sequence. Such kits comprise, in some instances, at least one polynucleotide that hybridizes to at least one of the methylation biomarker sequences described herein and at least one reagent for detection of gene methylation. Reagents for detection of methylation include, e.g., sodium bisulfate, polynucleotides designed to hybridize to sequence that is the product of a marker sequence if the marker sequence is not methylated (e.g., containing at least one C-U conversion), and/or a methylation-sensitive or methylation-dependent restriction enzyme. In some cases, the kits provide solid supports in the form of an assay apparatus that is adapted to use in the assay. In some instances, the kits further comprise detectable labels, optionally linked to a polynucleotide, e.g., a probe, in the kit. In some embodiments, the kit further comprises a process unit to obtain a weighted sum score as described herein.

Optionally, one or more detectably-labeled polypeptides capable of hybridizing to the amplified portion are also included in the kit. In some embodiments, the kits comprise sufficient primers to amplify the target DNA sequences described herein, and optionally include detectably-labeled polynucleotides capable of hybridizing to each amplified DNA region or portion thereof. The kits further can comprise a methylation-dependent or methylation sensitive restriction enzyme and/or sodium bisulfite.

In some embodiments, the kits comprise sodium bisulfite, primers and adapters for whole target genes amplification, and polynucleotides (e.g., detectably-labeled polynucleotides) to quantify the presence of the converted methylated and or the converted unmethylated sequence of at least one cytosine from a DNA region of an epigenetic biomarker described herein.

In some embodiments, the kits comprise methylation sensing restriction enzymes, primers and adapters for whole target genes amplification, and polynucleotides to quantify the number of copies of at least a portion of a DNA region of an epigenetic marker described herein. In some embodiments, the kits comprise a methylation binding moiety and one or more polynucleotides to quantify the number of copies of at least a portion of a DNA region of a marker described herein.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

Although the present disclosure has been described with an exemplary embodiment, various changes and modifications may be suggested to one skilled in the art. It is intended that the present disclosure encompass such changes and modifications as fall within the scope of the appended claims.

### EXAMPLE

### Materials and Methods

### Sample Preparation

Blood samples were collected using an ETDA-K2 tube and PAXgene Blood ccfDNA (circulating cell-free DNA) tube (Qiagen, Hilden, Germany, 768165) designed specifically for in vitro diagnostic ccfDNA testing. The samples collected using ETDA-K2 tube (BD, Plymouth, UK, 367525) were immediately centrifuged at 2000×g for 10 min at 4° C. Within 2 h, the supernatant from each sample was transferred to a new centrifuge tube and centrifuged at 6000×g for 30 min at 4° C. and subsequently stored at −80° C. Samples collected using the PAXgene Blood ccfDNA tube were kept at room temperature (15-25° C.) until use within 3 days, and subsequently centrifuged at 2000×g for 10 min at 4° C. followed by 6000×g for 30 min at 4° C. for plasma separation. The plasma of each sample was split into 1.6 mL aliquots and immediately frozen at −80° C. until further use.

### The Cancer Genome Atlas Portal

The data of the Western cohort are based on data generated by The Cancer Genome Atlas (TCGA) Research Network from Genomic Data Commons (GDC) data portal. The Cancer Genome Atlas (TCGA) is a collaboration between the National Cancer Institute (NCI) and the National Human Genome Research Institute (NHGRI) that has generated comprehensive, multidimensional maps of the key genomic changes in 33 types of cancer. The TCGA dataset, comprising more than two petabytes of genomic data, is now accessible to the cancer research community to improve the prevention, diagnosis and treatment of cancer.

### Genomic DNA Extraction

Genomic DNA from matched pairs of primary tumors and adjacent colorectal tissues from the same patient was extracted using the QIAamp DNA Mini Kit (Qiagen, Bonn, Germany, Cat. No. 51306) according to manufacturer's instruction. After DNA quantification, the purity was verified by measuring the A260/A280 ratio (range 1.8 to 2.0) using a NanoDrop ND-1000 spectrophotometer (NanoDrop Technologies Inc, Wilmington, DE, USA). 25

### Manual Circulating Cell-Free DNA Extraction

Circulating cell-free DNA (cfDNA) from plasma samples was extracted using the MagMAX Cell-Free DNA Isolation Kit (Thermo Fisher Scientific, Austin, TX, USA) or Catch-cfDNA Serum/Plasma kit (CatchGene, New Taipei City, Taiwan, R.O.C.) according to the manufacturer's recommended protocol. The cefDNA samples had clear fragment size peaks between 140 and 200 bp. The DNA Isolation kit provided the highest yield and low molecular weight fractions. The plasma was isolated immediately from 10 mL of peripheral blood within 2 h. After DNA quantification, the purity was verified by measuring the A260/A280 ratio (range 1.8 to 2.0) using a NanoDrop ND-1000 spectrophotometer (NanoDrop Technologies, Inc., Wilmington, DE, USA).

### Automatic Circulating Cell-Free DNA Extraction and Bisulfite Conversion by KingFisher™ Duo Prime

An automated process for ccfDNA extraction and bisulfite conversion on the KingFisher™ Duo Prime purification system (ThermoFisher Scientific, Singapore) was applied according to the manufacturer's instructions. This process fully automates magnetic bead-based DNA extraction of up to six samples simultaneously. The workflow was adapted as described in the instruction manual supplied with the MagMAX™ cell-free DNA isolation kit (ThermoFisher Scientific, Austin, TX, USA, A29319). The cefDNA was extracted from 1.6 mL of plasma and eluted in 60 μL of molecular biology-grade water (Corning, NY, USA, 46-000-CM). The bisulfite conversion cleanup was also performed on this machine for the semiautomatic assay. The automated protocol for the bisulfite conversion cleanup was developed with the instruction manual supplied with the EZ-96 DNA Methylation-Lightning™ MagPrep Kit (Zymo Research, Irvine, CA, USA, D5046). The extracted ccfDNA was incubated with sodium bisulfate (6 M) and hydroquinone (10 mM) in a 60° C. incubator for 30 min following by the automated process. We used 60 μL of ccfDNA for bisulfite conversion, and the bisulfite-converted cefDNA was eluted in 100 μL of molecular biology-grade water. The automated sample process was performed using a 24 deep-well plate (ThermoFisher Scientific, Vantaa, Finland, 95040470). The eluted bisulfite-converted ccfDNA was immediately used for methylation-specific real-time PCR.

### Automatic Circulating Cell-Free DNA Extraction Using LabTurbo 24C

The automated ccfDNA extraction process was performed using the LabTurbo 24 Compact System (Taigen Bioscience Co., Taipei, Taiwan) according to the manufacturer's instructions. The workflow followed the instruction manual supplied with the Labturbo Circulating DNA mini kit (Cat No. AIOLCD1600, Taigen Bioscience Co., Taipei, Taiwan), with full automation of vacuum-based DNA extraction of up to 24 samples simultaneously. The ccfDNA was extracted from 1.6 mL of plasma and eluted in 60 μL of molecular biology-grade water (46-000-CM, Corning, NY, USA).

### MethylationEPIC BeadChip Array for Genome-Wide Methylation Analysis

The MethylationEPIC BeadChip (EPIC) array covers 850,000 CpG sites, including >90% of the CpGs and 99% Refseq genes from HM450 and an additional 413,743 CpGs. The EPIC array has been validated in comparison to the 450K platform for blood samples. The genome-wide methylation analysis was performed using the Infinium® MethylationEPIC BeadChip array (Illumina, San Diego, CA, USA). Bisulfite conversion was performed for 500 ng of DNA using the EpiTect Fast DNA Bisulfite Kit (QIAGEN, Bonn, Germany, Cat. No. 59826) according to the manufacturer's instructions. Methylation scores for each CpG site are represented as “beta” values ranging from 0 (unmethylated) to 1 (fully methylated) by determining the ratios of the methylated signal intensities to the sums of the methylated and unmethylated signal outputs. Infinium MethylationEPIC BeadChip data were analyzed using GenomeStudio Methylation Module version 2011.1. The Infinium MethylationEPIC BeadChip employs both Infinium I and Infinium II assays. The Infinium I assay design employs 2 bead types per CpG locus, 1 each for the methylated and unmethylated states. The Infinium II design uses 1 bead type, with the methylated state determined at the single base extension step after hybridization (right panel). A differentially methylated CpG heatmap of the target genes was visualized by a heatmap using heatmapper software. A gradient-scale heatmap was used to visualize the DNA methylation level from low to high.

Probe-Based Quantitative Methylation-Specific PCR (qMSP)

After bisulfite conversion of DNA, which was done according to the manufacturer's recommended protocol, the DNA methylation levels of TMEM240, MROH6, BEND5, and SMAD3 were measured using TaqMan quantitative methylation-specific PCR (qMSP) with a LightCycler 96 (Roche Applied Science, Penzberg, Germany). qMSP was performed using the SensiFAST™ Probe No-ROX Kit (Bioline, London, UK, Cat. No. BIO-86020) with specific primers and methyl-TaqMan probes of candidate genes. Normalized DNA methylation values, which were calibrated to the control group, were obtained using LightCycler Relative Quantification software (Version 1.5, Roche Applied Science). The beta-actin (ACTB) gene was used as methylation-independent DNA control. The primers/probes for the ACTB gene were designed without the CpG site (as a control for input DNA). The primers/probes for candidate genes were designed on their methylated promotor regions, especially on the identified differential regions between normal and tumor tissues. According to the sequencing results, only when all CpG sites are methylated can a successful PCR reaction occur. The target genes were considered hypermethylated when the methylation level relative to that of the ACTB gene was at least 2-fold higher in the colorectal tumor compared with the paired normal colorectal tissue sample. The specificity of the candidate gene methylation end products was confirmed by bisulfite sequencing. The primers and probes used for qMSP are listed in Table 1.

### Statistical Analysis

The Pearson's chi-squared test, Mann-Whitney U test, Wilcoxon test and Spearman's rank correlation analyses were performed using SPSS (IBM, Armonk, NY, USA). The Pearson's chi-squared test was used to compare colorectal cancer patients in terms of candidate gene methylation, RNA expression and other clinical data. The paired-sample Wilcoxon test and t-test was used to compare differences in DNA methylation between tumors and matched adjacent normal tissues, different cancer types, as well as in candidate ccfDNA methylation between surgery treatment in colorectal cancer patients. The Spearman's rank correlation was adopted to analyze the methylation levels of the tumor and plasma samples.

To assess multiple biomarkers, Kang's nonparametric stepwise classification method was employed to evaluate the accuracy in identifying colorectal cancer patients when the proposed gene biomarkers were used. In addition to the accuracy, other commonly used measures for evaluating the classification, such as the area under the receiver operating characteristic curve (AUC), sensitivity, specificity, false-positive rate and false-negative rate, were also reported.

### Example 1 Methylation Status of Target DNA Sequence in Colorectal Cancer Tissue

The β value for Illumina Methylation 450K array-based data was generated from The Cancer Genome Atlas (TCGA) Research Network. The target nucleic acid and genes were selected when β value from normal tissues is less than 0.15; Δβ value (the value of Tumor subtracts that of normal tissues) is higher than 0.5 or lower than 0.25. The methylation statuses Δβ value (T)of the target DNA sequences are shown in Table 2.

TABLE 2

| | MROH6 | TMEM240 | BEND5 | SMAD3 |
|---|---|---|---|---|
| Colorectal cancer | 0.50 | 0.73 | 0.53 | 0.09 |

β value (T), methylation status of tumor tissues; β value(T)≥0.5 will be calculated as hypermethylation biomarkers and β value(T)≤0.25 will be calculated as hypomethylation biomarker.

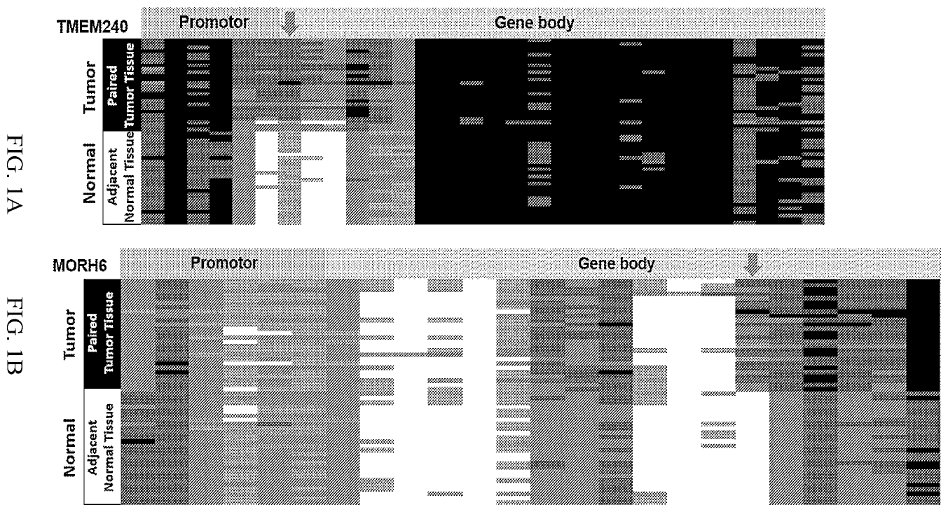

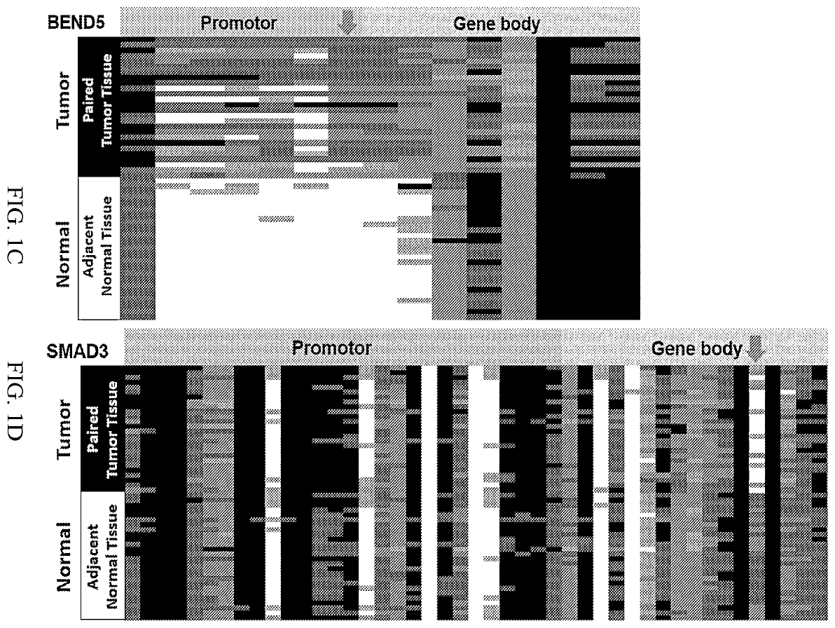

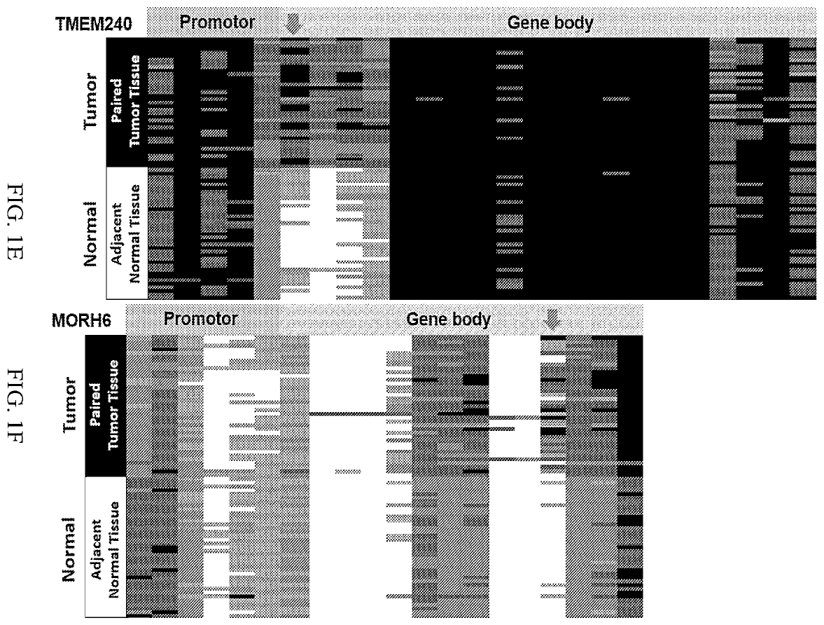

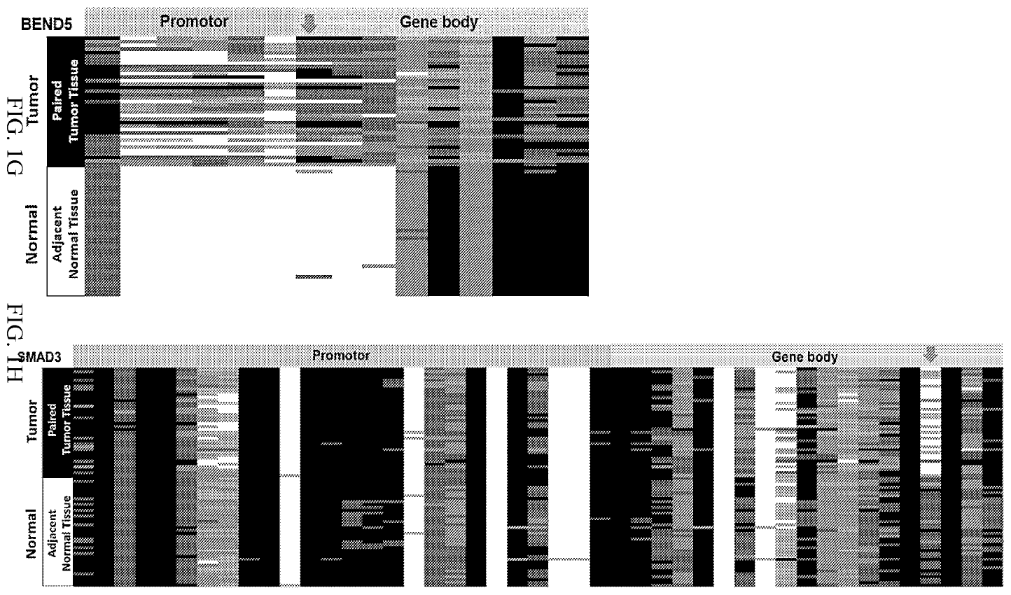

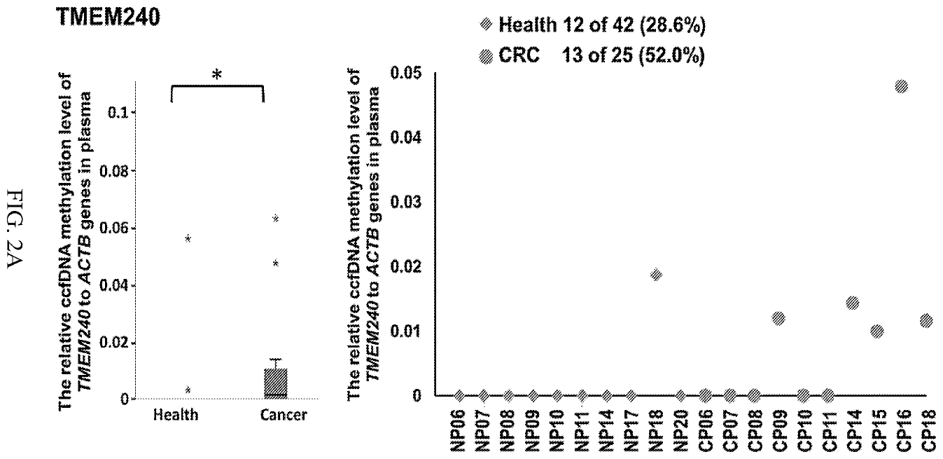

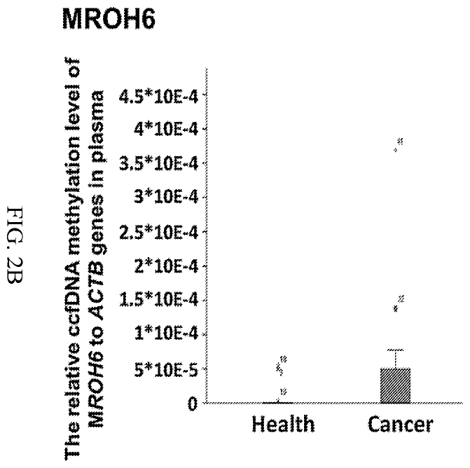

FIG. 1 shows the difference of methylation status (β value) of target nucleic acid and genes between tumor tissues and adjacent normal tissues (n=97). Darker color indicates tissues with higher methylation status according to Illumina Methylation 450K array-based data.

### Example 2 Early Detection of Methylation Status of Epigenetic Biomarkers of Target Genes in Plasma Samples of Healthy Subjects and Colorectal Cancer Patients

The circulating cell-free DNA was extracted from plasma. Briefly, 3.5 mL of plasma was isolated immediately from 10 mL of peripheral blood. After circulating cell-free DNA (cfDNA) was extracted from plasma that obtained from colorectal cancer patients and healthy subjects, cfDNA was performed by bisulfite conversion. Probe-based methylation specific real-time PCR (qMSP) was used for cfDNA methylation analyses.

Data obtained from the qMSP assays was processed according to the following criteria. If the Cₜ value of qMSP was less than 45 cycles for circulating methylated TMEM240 gene; less than 40 cycles for circulating methylated MROH6 gene; less than 45 cycles for circulating methylated BEND5; higher than 45 cycles for circulating methylated SMAD6, a score was defined as 1, respectively. Otherwise, a score was defined as 0.

The average of the total scores of qMSP in TMEM240, MROH6, and BEND5 was higher than 0.2, the human subject was defined as CRC patient.

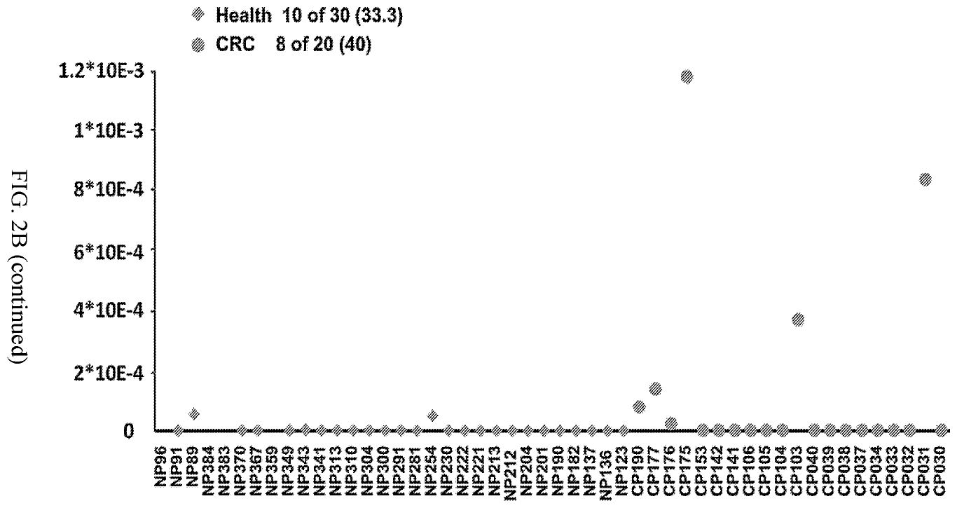

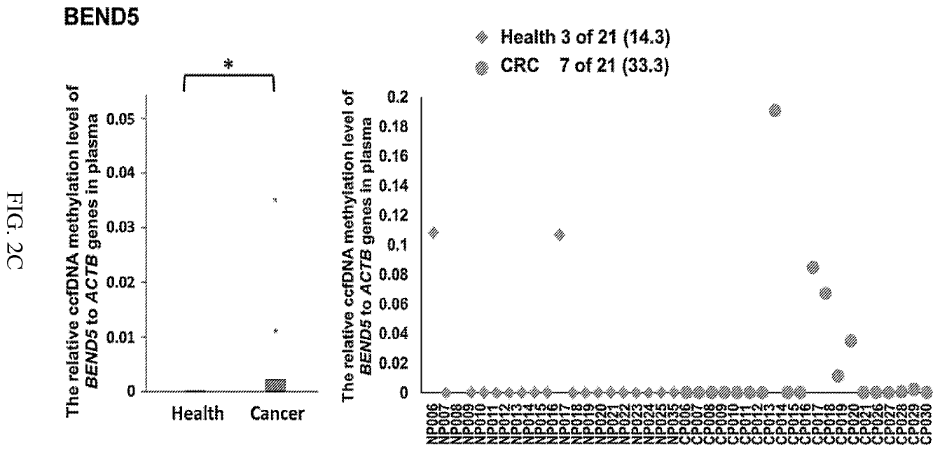

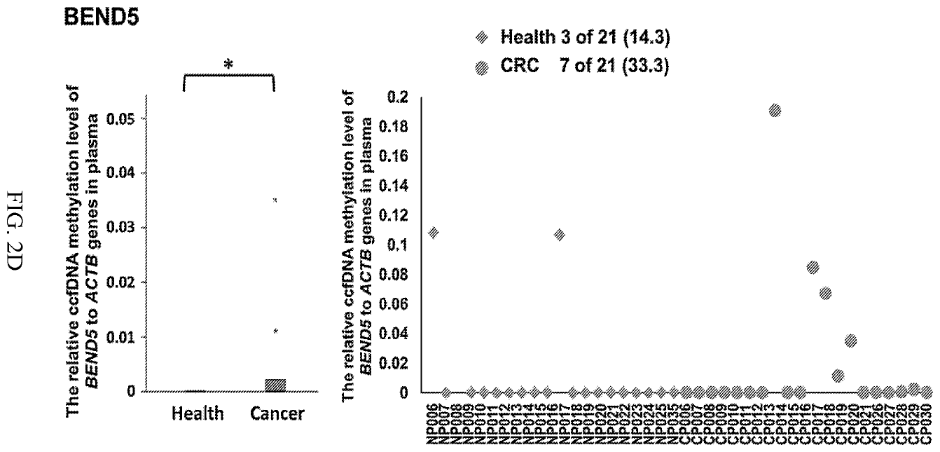

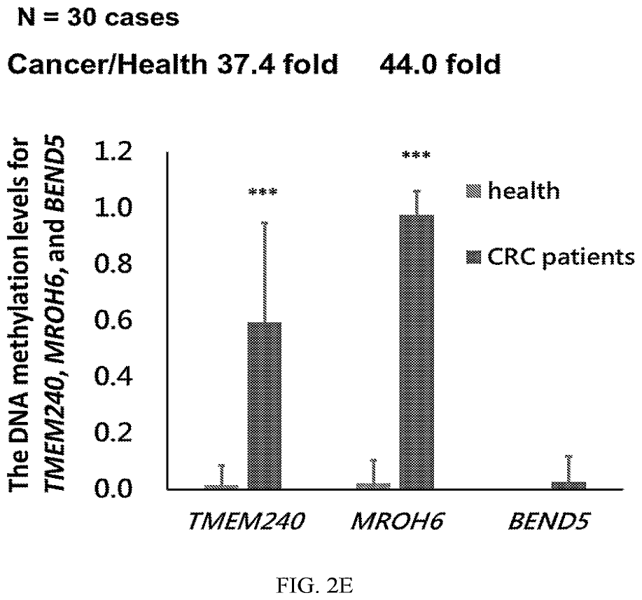

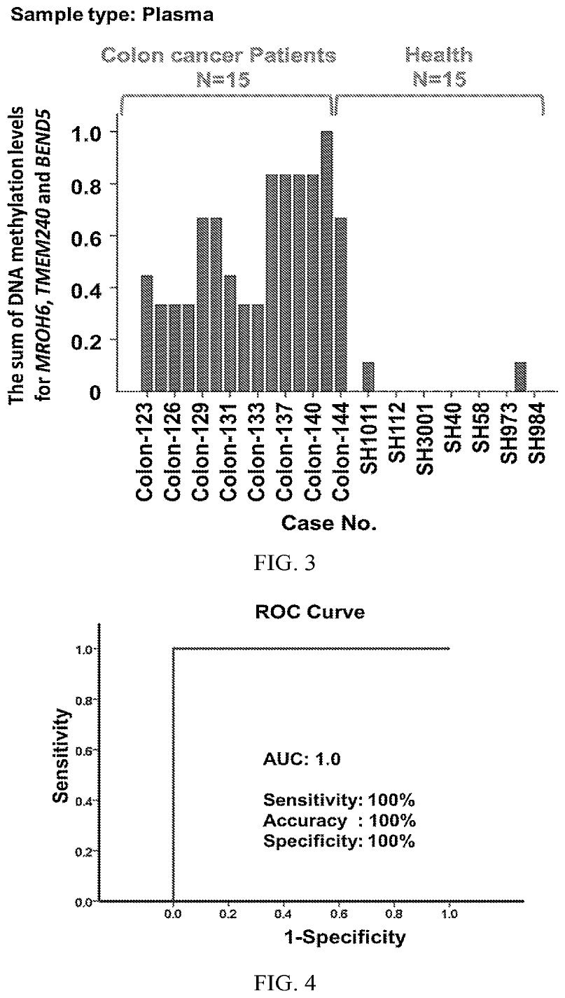

FIG. 2 shows the difference in early detection of the methylation status of epigenetic biomarkers of target genes in plasma samples of healthy subjects and colorectal cancer patients. FIG. 3 shows the sum of DNA methylation levels (scores) of epigenetic biomarkers of target genes in plasma samples of healthy subjects and colorectal cancer patients. In addition, Receiver Operating Characteristic (ROC) Curve Analysis as shown in FIG. 4 indicates the early detection of the methylation status of epigenetic biomarkers of target genes in colorectal cancer patients and healthy subjects.

1. A method for detecting a predisposition to colorectal cancer or predicting likelihood, treatment response, prognosis or recurrence of colorectal cancer in a subject, comprising (a) providing a biological sample from the subject, and (b) determining the methylation status of a target DNA sequence comprising MROH6 or a fragment thereof in the biological sample, wherein the presence of hypermethylation in MROH6 or a fragment of the subject is indicative of the predisposition to, likelihood, poor treatment response, poor prognosis or recurrence of, colorectal cancer.

2. The method of claim 1, wherein the biological sample is a tissue, cell, blood, urine, serum, plasma, stool, ascites, sputum, saliva, gastric juice, bile, or oral mucosa.

3. The method of claim 25, wherein the method further comprises a step of defining a score as 1 if the presence of hypermethylation or hypomethylation of each gene or a fragment thereof and, defining a score as 0 if the absence of hypermethylation or hypomethylation of each gene or a fragment thereof, and summing the score.

4. The method of claim 1, wherein the presence of hypermethylation or hypomethylation in the target DNA sequence of the subject is determined by comparing the methylation status of the target DNA sequence to a methylation status of a control DNA sequence.

5. The method of claim 1, wherein the methylation status is determined by a polymerase chain reaction.

6. (canceled)

7. (canceled)

8. The method of claim 1, wherein a target DNA sequence methylation specific primer for MROH6 or a fragment thereof is used in the methylation determination; and the target DNA sequence methylation specific primer for MROH6 or a fragment thereof has an identity of about 85% to a sequence selected from the group consisting of SEQ ID NOs: 5, and 6.

9. The method of claim 1, wherein a target DNA sequence methylation specific probe for MROH6 or a fragment thereof is used in the methylation determination; and the target DNA sequence methylation specific probe for MROH6 or a fragment thereof has an identity of about 85% to a sequence of SEQ ID NO: 7.

10. (canceled)

11. The method of claim 25, wherein the target DNA sequence comprises any of the following combinations of the DNA sequences: TMEM240 or a fragment thereof and MROH6 or a fragment thereof; TMEM240 or a fragment thereof, MROH6 or a fragment thereof and BEND5 or a fragment thereof; TMEM240 or a fragment thereof, MROH6 or a fragment thereof, BEND5 or a fragment thereof and SMAD3 or a fragment thereof; TMEM240 or a fragment thereof, BEND5 or a fragment thereof and SMAD3 or a fragment thereof; TMEM240 or a fragment thereof, MROH6 or a fragment thereof, and SMAD3 or a fragment thereof; MROH6 or a fragment thereof and BEND5 or a fragment thereof; and MROH6 or a fragment thereof, BEND5 or a fragment thereof and SMAD3 or a fragment thereof.

12. (canceled)

13. (canceled)

14. The method of claim 25, wherein a target DNA sequence methylation specific primer for TMEM240 or a fragment thereof, BEND5 or a fragment thereof, or SMAD3 or a fragment thereof or a combination thereof is used in the methylation determination, and wherein the target DNA sequence methylation specific primer for TMEM240 or a fragment thereof has an identity of about 85% to a sequence selected from the group consisting of SEQ ID NOs: 1, 2, and 3; the target DNA sequence methylation specific primer for BEND5 or a fragment thereof has an identity of about 85% to a sequence selected from the group consisting of SEQ ID NOs: 8 to 11; and the target DNA sequence methylation specific primer for SMAD3 or a fragment thereof has an identity of about 85% to a sequence selected from the group consisting of SEQ ID NOs: 14 and 15.

15. The method of claim 25, wherein a target DNA sequence methylation specific probe for TMEM240 or fragment thereof, BEND5 or a fragment thereof, or SMAD3 or a fragment thereof or a combination thereof is used in the methylation determination, and wherein the target DNA sequence methylation specific probe for TMEM240 or a fragment thereof has an identity of about 85% to a sequence of SEQ ID NO: 4; the target DNA sequence methylation specific probe for BEND5 or a fragment thereof has an identity of about 85% to a sequence selected from the group consisting of SEQ ID NOs: 12 and 13; and the target DNA sequence methylation specific probe for SMAD3 or a fragment thereof has an identity of about 85% to a sequence of SEQ ID NO: 16.

16. The method of claim 25, wherein determining the methylation status further comprises a step of measuring the specificity and sensitivity by a weighted sum score analysis.

17. An isolated nucleic acid molecule having a sequence selected from the group consisting of: SEQ ID Nos: 1 to 16.

18. A kit for detecting a predisposition to colorectal cancer or predicting likelihood, treatment response, prognosis or recurrence of colorectal cancer in a subject, which comprises a target DNA sequence methylation specific primer pair for detection of a methylation status of a target DNA sequence comprising MROH6 or a fragment thereof.

19. The kit of claim 18, further comprising a target DNA sequence methylation specific probe for detection of a methylation status of a target DNA sequence comprising MROH6 or a fragment thereof.

20. The kit of claim 18, wherein the target DNA sequence methylation specific primer pair for MROH6 or a fragment thereof comprises SEQ ID NOs: 5 and 6.

21. The kit of claim 18, the target DNA sequence methylation specific probe for MROH6 or a fragment thereof comprises SEQ ID NO: 7.

22. The kit of claim 18, wherein the target DNA sequence further comprises TMEM240 or a fragment thereof, BEND5 or a fragment thereof, or SMAD3 or a fragment thereof, and the kit further comprises one or more target DNA sequence methylation specific primer pairs for detection of a methylation status of TMEM240 or a fragment thereof, BEND5 or a fragment thereof, or SMAD3 or a fragment thereof, or any combination thereof.

23. The kit of claim 18, wherein the target DNA sequence methylation specific primer pair for TMEM240 or a fragment thereof comprises SEQ ID NOs: 1 and 2 or SEQ ID NOs: 1 and 3; the target DNA sequence methylation specific primer pair for BEND5 or a fragment thereof comprises SEQ ID Nos. 8 and 9 or SEQ ID NOs: 10 and 11; the target DNA sequence methylation specific primer pair for SMAD3 or a fragment thereof comprises SEQ ID Nos. 14 and 15.

24. The kit of claim 18, wherein the kit further comprises one or more target DNA sequence methylation specific probe for detection of a methylation status of TMEM240 or a fragment thereof, BEND5 or a fragment thereof, or SMAD3 or a fragment thereof, or any combination thereof, and wherein the target DNA sequence methylation specific probe for TMEM240 or a fragment thereof comprises SEQ ID NO: 4; the target DNA sequence methylation specific probe for BEND5 or a fragment thereof comprises SEQ ID NO: 12 or 13; and the target DNA sequence methylation specific probe for SMAD6 or a fragment thereof comprises SEQ ID NO: 16.

25. The method of claim 1, wherein the target DNA sequence further comprises one or more DNA sequences selected from the group consisting of TMEM240 or a fragment thereof, BEND5 or a fragment thereof and SMAD3 or a fragment thereof, or any of combinations thereof; wherein the presence of hypermethylation in TMEM240 or a fragment, hypermethylation in BEND5 or a fragment, or hypomethylation in SMAD3 or a fragment, of the subject is indicative of the predisposition to, likelihood, poor treatment response, poor prognosis or recurrence of, colorectal cancer.


SEQUENCE LISTING (16 sequences)

SEQ ID NO: 1 - DNA, 27 bp, Artificial Sequence
  primer
tttagaattatgaagattatggtgttc

SEQ ID NO: 2 - DNA, 20 bp, Artificial Sequence
  primer
aaaactcaacatcgaaccga

SEQ ID NO: 3 - DNA, 22 bp, Artificial Sequence
  primer
cgaccccgcccgatatccataa

SEQ ID NO: 4 - DNA, 27 bp, Artificial Sequence
  probe
tttagaattatgaagattatggtgttc

SEQ ID NO: 5 - DNA, 25 bp, Artificial Sequence
  primer
ggtgagtttttgatttgtaattgtc

SEQ ID NO: 6 - DNA, 23 bp, Artificial Sequence
  primer
atctcgtaccgctactactacgc

SEQ ID NO: 7 - DNA, 28 bp, Artificial Sequence
  probe
gtcgggggttgttgattttagtagcgtt

SEQ ID NO: 8 - DNA, 19 bp, Artificial Sequence
  primer
gtttgggttttggggagtc

SEQ ID NO: 9 - DNA, 19 bp, Artificial Sequence
  primer
gatcgaacaactcaacccg

SEQ ID NO: 10 - DNA, 20 bp, Artificial Sequence
  primer
gtttttgtgcggtttttgga

SEQ ID NO: 11 - DNA, 20 bp, Artificial Sequence
  primer
aaccgcgaacgaaaactaaa

SEQ ID NO: 12 - DNA, 20 bp, Artificial Sequence
  probe
cgaaaataaaaatccgacga

SEQ ID NO: 13 - DNA, 20 bp, Artificial Sequence
  probe
ttgttacgcgttgttcgtgt

SEQ ID NO: 14 - DNA, 25 bp, Artificial Sequence
  primer
gaataaggtcgttagttattatcgt

SEQ ID NO: 15 - DNA, 22 bp, Artificial Sequence
  primer
aatcaaatctacccgaatcgaa

SEQ ID NO: 16 - DNA, 36 bp, Artificial Sequence
  probe
gaaagaaagaaagaaagtaaattttatttttaagcg